(19) United States
(12) Patent Application Publication
(10) Pub. No.: US 2005/0135979 A1
(43) Pub. Date: Jun. 23, 2005

(54) Device for deodorizing a sink drain and method therefor

(76) Inventor: Steve Gootter, Scottsdale, AZ (US)

Correspondence Address: WEISS & MOY PC, 4204 NORTH BROWN AVENUE, SCOTTSDALE, AZ, 85251, US

(21) Appl. No.: 10/945,792

(22) Filed: Sep. 21, 2004

Related U.S. Application Data
(60) Provisional application No. 60/531,057, filed on Dec. 18, 2003.

(51) Int. Cl. A61L 2/18; E03D 9/02

(52) U.S. Cl. 422/266; 4/222; 4/231

(57) ABSTRACT

A device for deodorizing a sink drain that allows the sink drain to remain substantially entirely open. Preferably, the device would deodorize sink drains. Further preferably, the device would treat sink drain clogs. Further preferably, the device would both deodorize the sink drain and treat sink drain clogs. Still further preferably, the device would have a wear indicator to signal the user when a new device is needed.

### CROSS-REFERENCE TO RELATED APPLICATION

This application claims priority to a corresponding provisional application U.S. Ser. No. 60/531,057, filed Dec. 18, 2003 in the names of the applicants of this application.

### BACKGROUND OF THE INVENTION

1. Field of the Invention

This invention relates generally to sink drains and, more specifically, to a device capable of deodorizing a sink drain area and/or removing clogs inside the sink drain.

2. Description of Prior Art

Many sinks have been equipped with a garbage disposal unit. The garbage disposal unit grinds large food and waste particles into smaller particles that are then diverted into the drain pipe. The garbage disposal unit, however, may not be able to divert all of the waste particles, dirt, oil, and grease into the drain pipe. Build-up of bacteria, foul odors, and clogs may then begin to develop in the sink drain due to the dirt, oil, grease, and food particles that may have become trapped within it. Additionally, sink drains without a garbage disposal unit also tend to accumulate food particles which can cause a build-up of bacteria, causing both a foul odor as well as drain clogs.

One way by which people have attempted to combat the development of bacterial build-up and odors is by using antibacterial and deodorizing sprays. Although the sprays may eliminate some of the bacteria and the odors, they are easily washed away with the liquids poured into the drain.

Another way that people, especially those within the food process industry, have attempted to combat bacterial build-up and odors is to insert a drain sanitizing article into the drain. U.S. Pat. No. 6,197,321 issued to Richter et al. discloses a sanitizing article housing preferably molded with a handle as a one piece unit. The handle traverses and partially obstructs the inner opening of the housing so as to allow the handle to rest on a stand pipe which projects from a drain.

A problem arises, however, when this sanitizing article is placed within a sink equipped with a garbage disposal unit. Because the handle traverses the inner opening of the housing, a user is limited to the size and the type of debris that he/she wishes to place into the garbage disposal unit. The user would only be able to place debris capable of passing through the spaces unimpeded by the handle into the garbage disposal unit. And if the user were to wash a long pasta noodle or other similarly shaped debris down the drain opening, the noodle could get caught on the handle.

Furthermore, U.S. Pat. No. 4,318,193 issued to Bayer et al. discloses a flexible deodorant ring holder. The Bayer et al. device comprises a flexible arcuate shaped holder formed by a flexible arcuate outer rim, a plurality of radially extending rib members, and a flexible arcuate inner rim. The device therefore has an adjustable outside diameter for holding a rigid deodorant ring having a fixed outside diameter.

The configuration of the Bayer et al. device, however, would actually contribute to the problem of odors rather than solve it. Particularly, food particles and other debris may become lodged between or within the rib members of the flexible holder. Not only will the trapped food particles and debris cause odors, but they are also unpleasant to the eye. Furthermore, the flexible holder obstructs a substantial portion of the sink opening, thereby limiting the size and the type of debris that a user may wish to place into the underlying garbage disposal unit. Again, the user would only be able to place debris capable of passing through the spaces unimpeded by the rib members of the flexible holder into the garbage disposal unit. And if the user were to wash a long pasta noodle or other similarly shaped debris down the drain opening, the noodle could get caught on the flexible rib members of the flexible holder.

Therefore a need existed to provide a device for deodorizing a sink drain that allows the sink drain to remain substantially entirely open. Preferably, the device would remove sink drain clogs. Further preferably, the device would both deodorize the sink drain and remove sink drain clogs. Still further preferably, the device would have a wear indicator to signal the user when a new device is needed.

### SUMMARY OF THE INVENTION

It is an object of the present invention to provide a device for deodorizing a sink drain and method therefor that allows the sink drain to remain substantially entirely open.

It is a further object of the present invention to provide a device for deodorizing a sink drain and method therefor that removes sink drain clogs.

It is a further object of the present invention to provide a device for deodorizing a sink drain and method therefor that both deodorizes the sink drain and removes sink drain clogs.

It is a still further object of the present invention to provide a device for deodorizing a sink drain and method therefor that employs a wear indicator to signal a user when a new device is needed.

### BRIEF DESCRIPTION OF THE PREFERRED EMBODIMENTS

In accordance with one embodiment of the present invention, a device for deodorizing a sink drain is disclosed. The device for deodorizing a sink drain comprises at least one deodorizing ring, the at least one deodorizing ring being dimensioned to be substantially flush with an interior wall of the sink drain while at the same time defining an aperture dimensioned to allow the sink drain to remain substantially entirely open.

In accordance with another embodiment of the present invention, a device for deodorizing a sink drain is disclosed. The device for deodorizing a sink drain comprises at least one deodorizing ring, the at least one deodorizing ring being dimensioned to be coupled proximate an exterior portion of the sink drain while at the same time defining an aperture dimensioned to allow the sink drain to remain substantially entirely open.

In accordance with still another embodiment of the present invention, a method for deodorizing a sink drain is disclosed. The method for deodorizing a sink drain comprises, in combination, the steps of providing at least one deodorizing ring, the at least one deodorizing ring having a deodorizing agent and defining an aperture dimensioned to allow the sink drain to remain substantially entirely open, and positioning the at least one deodorizing ring such that the at least one deodorizing ring is substantially flush with an interior wall of the sink drain.

In accordance with yet another embodiment of the present invention, a method for removing clogs from a sink drain is disclosed. The method for removing clogs from a sink drain comprises, in combination, the steps of providing at least one deodorizing ring, the at least one deodorizing ring having a clog removing agent and defining an aperture dimensioned to allow the sink drain to remain substantially entirely open, and positioning the at least one deodorizing ring such that the at least one deodorizing ring is substantially flush with an interior wall of the sink drain.

In accordance with yet another embodiment of the present invention, a method for deodorizing a sink drain is disclosed. The method for deodorizing a sink drain comprises, in combination, the steps of providing at least one deodorizing ring, the at least one deodorizing ring containing a deodorizing agent being dimensioned to be coupled proximate an exterior portion of the sink drain while at the same time defining an aperture dimensioned to allow the sink drain to remain substantially entirely open, providing a refillable tray, the at least one refillable tray being dimensioned to house the at least one deodorizing ring, providing a plurality of legs having a first end and a second end, the first end of each leg being coupled to a portion of the refillable tray and the second end of each leg extending substantially downwardly from the at least one refillable tray, inserting the at least one deodorizing ring into the at least one refillable tray, and coupling the second end of each leg of the refillable tray to the sink drain so that the at least one deodorizing ring is supported above the sink drain.

The foregoing and other objects, features, and advantages of the invention will be apparent from the following, more particular, description of the preferred embodiments of the invention, as illustrated in the accompanying drawings.

### BRIEF DESCRIPTION OF THE DRAWINGS

FIG. 1 is a perspective view of a first embodiment of the device for deodorizing a sink drain consistent with the present invention. The first embodiment comprises a deodorizing ring.

FIG. 2 is a perspective cross-sectional view of the device of FIG. 1 being placed into a sink drain such that an outer surface of the device directly abuts an interior wall of the sink drain.

FIG. 3 is a top view of the device of FIG. 1 shown in use with the sink drain.

FIG. 4 is a perspective view of a second embodiment of a device for deodorizing a sink drain consistent with the present invention. The second embodiment comprises a refillable tray dimensioned to house at least one deodorizing ring.

FIG. 5 is a perspective view of the device of FIG. 4 having three deodorizing rings.

FIG. 6 is a perspective cross-sectional view of the device of FIG. 4 being placed into a sink drain such that an outer surface of the device directly abuts the interior wall of the sink drain.

FIG. 7 is a perspective view of a third embodiment of a device for deodorizing a sink drain consistent with the present invention. The third embodiment comprises a flexible crescent-shaped deodorizing ring. The flexible crescent-shaped deodorizing ring is shown in a contracted position.

FIG. 8 is a perspective view of a fourth embodiment of a device consistent with the present invention. The fourth embodiment comprises a hinged crescent-shaped deodorizing ring. The hinged crescent-shaped deodorizing ring is shown in a contracted position.

FIG. 9 is a perspective cross-sectional view of a fifth embodiment of a device for deodorizing a sink drain consistent with the present invention. The fifth embodiment comprises a flexible crescent-shaped refillable tray dimensioned to house at least one deodorizing ring. The device is shown as being squeezed and placed into a sink drain such that an outer surface of the device directly abuts the interior wall of the sink drain. A deodorizing ring is also shown being placed within the flexible crescent-shaped refillable tray.

FIG. 10 is a perspective view of a sixth embodiment of a device for deodorizing a sink drain consistent with the present invention. The sixth embodiment comprises a hinged crescent-shaped refillable tray dimensioned to house at least one deodorizing ring. The device is shown in a contracted position and three deodorizing rings are shown being placed within the hinged crescent-shaped refillable tray.

FIG. 11 is a perspective view of the hinged crescent-shaped deodorizing ring of FIG. 8 being placed within the hinged crescent-shaped refillable tray of FIG. 10. Both the hinged crescent-shaped deodorizing ring and the hinged crescent-shaped refillable tray are shown in a contracted position.

FIG. 12 is a top view of the device of FIG. 7 in use with a sink drain.

FIG. 13 is a perspective view of a seventh embodiment of a device for deodorizing a sink drain consistent with the present invention. The seventh embodiment comprises a deodorizing ring having legs dimensioned to support the deodorizing ring above the sink drain.

FIG. 14 is a top view of the device of FIG. 13 shown in use with a sink drain.

FIG. 15 is a perspective view of an eighth embodiment of a device for deodorizing a sink drain consistent with the present invention. The eighth embodiment comprises a refillable tray dimensioned to house at least one deodorizing ring and having legs dimensioned to support the refillable tray above the sink drain. A deodorizing ring is also shown being placed within the refillable tray.

FIG. 16 is a perspective view of the device of FIG. 15 being placed above the sink drain. A deodorizing ring is also shown being placed within the refillable tray.

FIG. 17 is a perspective view of a wear indicator shown integrally coupled to a deodorizing ring. The wear indicator is shown as being a color that fades as the deodorizing ring is diminished by use.

FIG. 18 is a perspective view of a wear indicator shown integrally coupled to a refillable tray. The wear indicator is shown as being a color that fades as the deodorizing ring is diminished by use.

### DETAILED DESCRIPTION OF THE PREFERRED EMBODIMENTS

The novel features believed characteristic of the invention are set forth in the appended claims. The invention will best be understood by reference to the following detailed description of illustrated embodiments when read in conjunction with the accompanying drawings, wherein like reference numerals and symbols represent like elements.

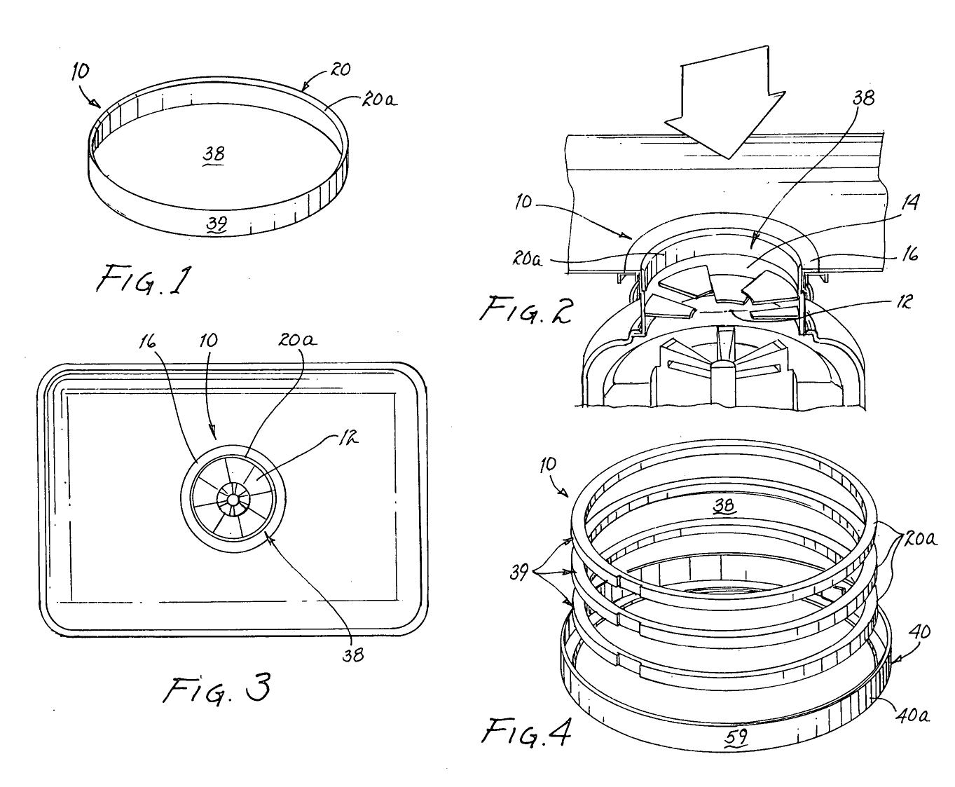

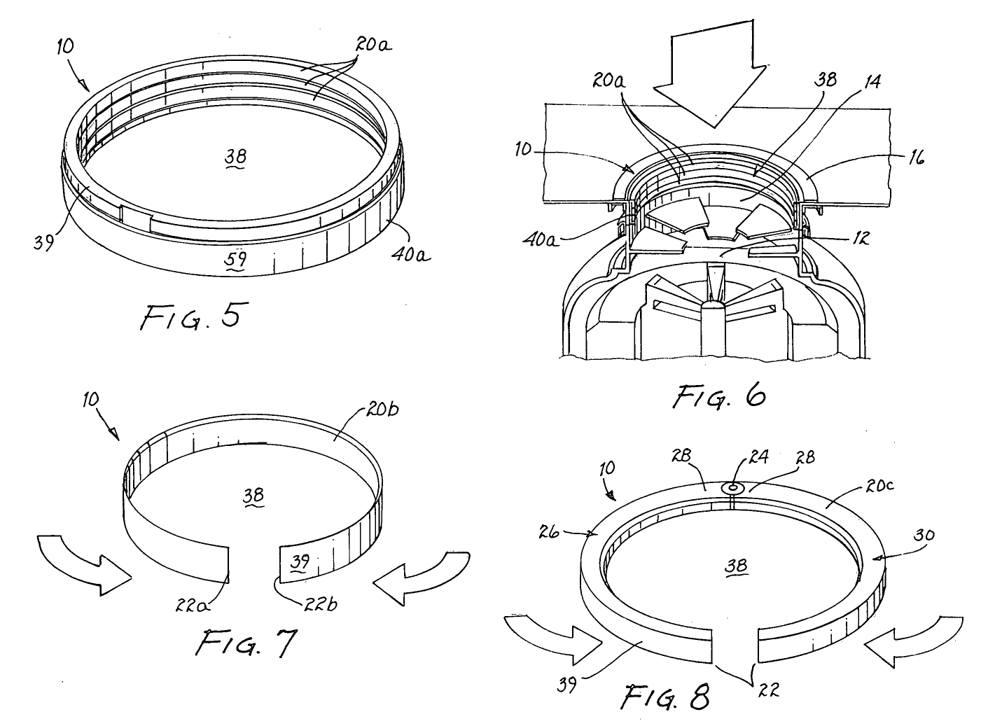

Referring to FIGS. 1-16, a device for deodorizing a sink drain, hereinafter device for deodorizing a sink drain 10, is disclosed. The device for deodorizing a sink drain 10 comprises at least one deodorizing ring 20. As shown in FIGS. 1-3, 7, 8, and 12-14, the device for deodorizing a sink drain 10 may comprise at least one deodorizing ring 20, or as shown in FIGS. 4-6, 9-11, 15, and 16, the device for deodorizing a sink drain 10 may further comprise a refillable tray 40 dimensioned to house the at least one deodorizing ring 20. Although FIGS. 4-6 and FIG. 10 show the refillable tray 40 as housing up to three deodorizing rings 20, it should be clearly understood that substantial benefit may be derived from the refillable tray 40 being dimensioned to house any number of deodorizing rings 20 so long as the refillable tray 40 is dimensioned to fit the sink drain 12.

Preferably, the device for deodorizing a sink drain 10 will have an outer diameter of between 8 cm and 10 cm, an inner diameter of between 7 cm and 9 cm, and a width of between 0.5 cm and 2.5 cm. Although these dimensions are preferred, it should be clearly understood that substantial benefit may be derived from alternate dimensions, provided that the device for deodorizing a sink drain 10 is dimensioned to fit a sink drain 12 and is dimensioned to allow the sink drain 12 to remain substantially entirely open. It should also be understood that although it is preferable that the device for deodorizing a sink drain 10 be used with a standard-sized kitchen sink drain 12, substantial benefit could be derived from alternative dimensions, even those that deviate substantially from the preferred dimensions in either direction, in order to use the device for deodorizing a sink drain 10 in sink drains 12 of varying sizes (e.g., bathroom sink drains, large industrial kitchen sink drains, etc.).

Referring now to FIGS. 1-12, the device for deodorizing a sink drain 10 may be dimensioned to be substantially flush with an interior wall 14 of the sink drain 12. Referring to FIGS. 1-3, 7, 8, and 12, the deodorizing ring 20 may be dimensioned such that an outer surface 39 (see FIGS. 1, 7, and 8) of the deodorizing ring 20 directly abuts the interior wall 14 of the sink drain 12. Similarly, as shown in FIGS. 4-6 and 9-11, the deodorizing ring 20 may be housed within a refillable tray 40 dimensioned such that an outer surface 59 of the refillable tray 40 directly abuts the interior wall 14 of the sink drain 12.

The deodorizing ring 20a (referred to generically as deodorizing ring 20) shown in FIGS. 1-5, 9, and 15 and the refillable tray 40a shown in FIGS. 4-6 (referred to generically as refillable tray 40) may have a substantially circular configuration. Alternatively, FIGS. 7-12 show deodorizing rings 20 and refillable trays 40 that are substantially crescent-shaped and that have outer diameters substantially larger than the inner diameter of the sink drain 12.

For example, FIGS. 7 and 12 disclose a substantially flexible crescent-shaped deodorizing ring 20b having a first grip end 22a (referred to generically as grip end 22) and a second grip end 22b (referred to generically as grip end 22). Similarly, FIG. 9 discloses a substantially flexible crescent-shaped refillable tray 40b having a first grip end 42a (referred to generically as grip end 42)(not shown) and a second grip end 42b (referred to generically as grip end 42)(not shown). When inserting the deodorizing ring 20b or the refillable tray 40b into the sink drain 12, the grip ends 22 of the deodorizing ring 20b or the grip ends 42 of the refillable tray 40b may be squeezed together, thereby causing the deodorizing ring 20b or the refillable tray 40b to contract. And by releasing the grip ends 22 of the deodorizing ring 20b or the grip ends 42 of the refillable tray 40b, the deodorizing ring 20b or the refillable tray 40b will expand and will be held in place within the sink drain 12.

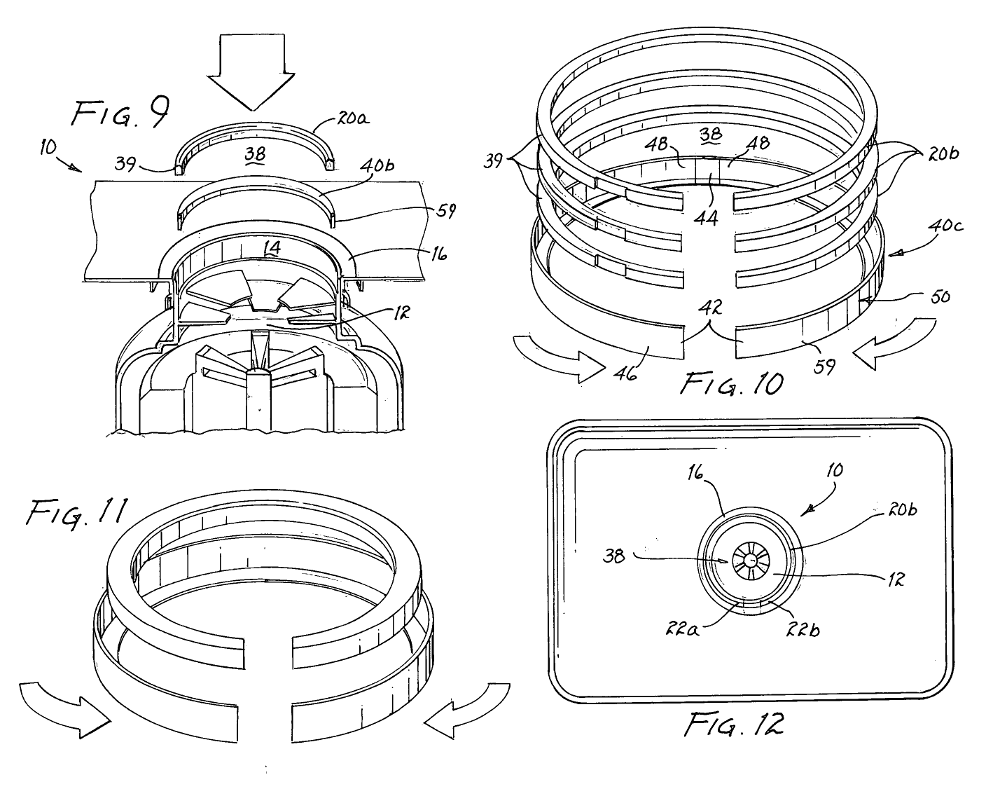

As a further example, FIG. 8 discloses a hinged crescent-shaped deodorizing ring 20c having a first portion 26 and a second portion 30. Both the first portion 26 and the second portion 30 have a hinge end 28 coupled to a hinge 24. Both the first portion 26 and the second portion 30 also have a grip end 22 which, when squeezed together, cause the deodorizing ring 20c to contract. While contracted, the deodorizing ring 20c may be inserted into the sink drain 12. Upon spreading apart or releasing the grip ends 22, the deodorizing ring 20c will expand and be held in place within the sink drain 12.

Similarly, FIGS. 10 and 11 disclose a hinged crescent-shaped refillable tray 40c having a first portion 46 and a second portion 50. Both the first portion 46 and the second portion 50 have a hinge end 48 coupled to a hinge 44. Both the first portion 46 and the second portion 50 also have a grip end 42 which, when squeezed together, cause the refillable tray 40c to contract. While contracted, the refillable tray 40c may be inserted into the sink drain 12. Upon spreading apart or releasing the grip ends 42, the refillable tray 40c will expand and be held in place within the sink drain 12.

Although it is preferred that the hinge 24 of the deodorizing ring 20c and the hinge 44 of the refillable tray 40c be one of a living hinge and a locking hinge, it should be clearly understood that substantial benefit may be derived from the use of a spring hinge.

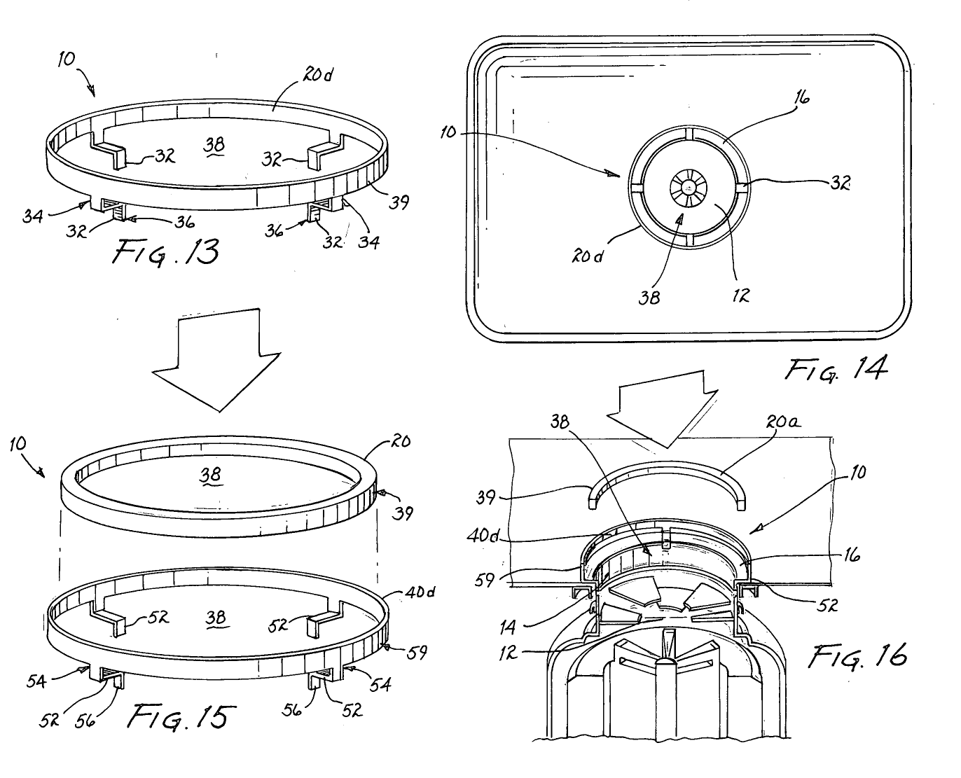

Referring now to FIGS. 13-16, the device for deodorizing a sink drain 10 may also be dimensioned to be coupled proximate an exterior portion 16 of the sink drain 12. FIGS. 13 and 14 show a deodorizing ring 20d having a plurality of legs 32. Each leg 32 has a first end 34 coupled to a portion of the deodorizing ring 20d and each leg 32 also has a second end 36 extending substantially downwardly from the deodorizing ring 20d. Each second end 36 is also dimensioned to couple the deodorizing ring 20d to the sink drain 12 so as to support the deodorizing ring 20d above the sink drain 12.

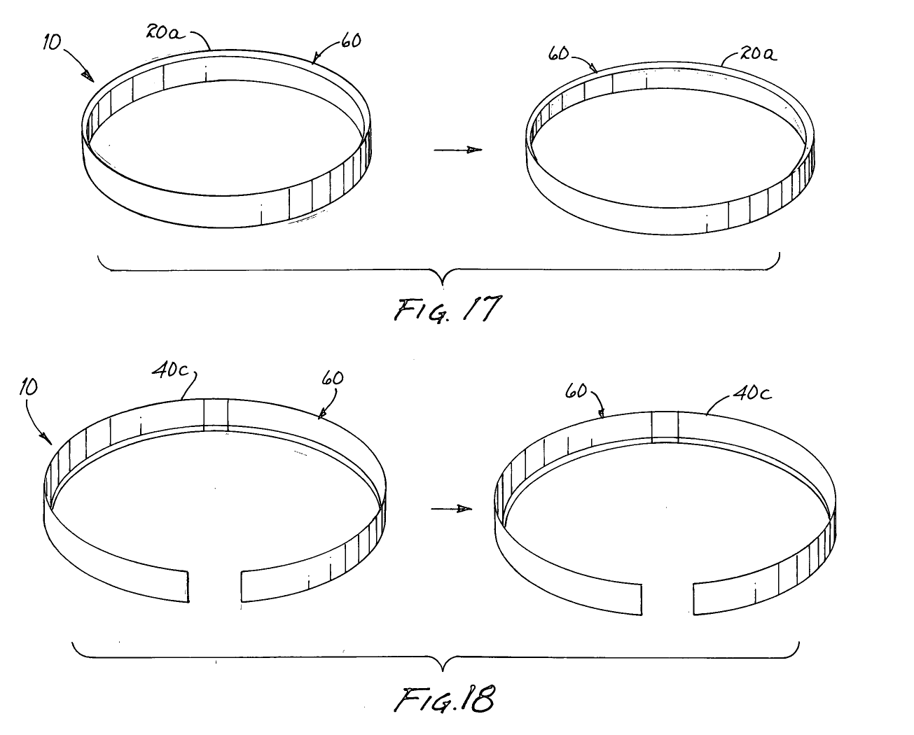

Similarly, FIGS. 15 and 16 show a refillable tray 40d having a plurality of legs 52. Each leg 52 has a first end 54 coupled to a portion of the refillable tray 40d and each leg 52 also has a second end 56 extending substantially downwardly from the refillable tray 40d. Each second end 56 is also dimensioned to couple the refillable tray 40d to the sink drain 12 so as to support the refillable tray 40d above the sink drain 12.

Although, in the embodiments of the device for deodorizing a sink drain 10 shown in FIGS. 13-16, it is preferred that deodorizing ring 20d have a plurality of legs 32 or that the refillable tray 40d have a plurality of legs 52, it should be clearly understood that substantial benefit may be derived from alternate configurations of the deodorizing ring 20d or the refillable tray 40d, so long as the deodorizing ring 20d or the refillable tray 40d is supported above the sink drain 12.

In order to combat odors, it is preferred that the device for deodorizing a sink drain 10 contain a deodorizing agent, such as a surfactant or caustic soda. In order to remove drain clogs, it is preferred that the device for deodorizing a sink drain 10 contain a clog removing agent, such as an enzyme or bleach.

It should be clearly understood that substantial benefit could be derived from: 1) a device for deodorizing a sink drain 10 directed to deodorizing drains in which each deodorizing ring 20 contains only a deodorizing agent; 2) a device for deodorizing a sink drain 10 directed to treating drain clogs in which each deodorizing ring 20 contains only a clog removing agent; or 3) a device for deodorizing a sink drain 10 directed to both deodorizing and treating clogs in which some deodorizing rings 20 contain a deodorizing agent while others contain a clog treating agent or in which each deodorizing ring 20 contains both a deodorizing agent and a clog treating agent.

It is also preferred that the device for deodorizing a sink drain 10 contain a fragrance.

As shown in FIGS. 17 and 18, it is further preferable that the device for deodorizing a sink drain 10 have a wear indicator 60 integrally coupled to at least one of the deodorizing ring 20 (see FIG. 17) and the refillable tray 40 (see FIG. 18). Preferably, the wear indicator 60 is integral to the deodorizing ring 20. The wear indicator 60 is adapted to signal a user to replace the device for deodorizing a sink drain 10 after an amount of wear on the device for deodorizing a sink drain 10. Presumably, the flow of water through the sink drain 10 will activate the wear indicator 60, although it should be understood that substantial benefit could be derived from a wear indicator 60 that utilizes alternative means for identifying the amount of wear.

As shown in FIG. 17, the wear indicator 60 may be a color that fades as the at least one deodorizing ring 20 is diminished by use. Alternatively, the wear indicator 60 may be a color that gradually changes to another color as the at least one deodorizing ring 20 is diminished by use. Instead of a color indicator, each deodorizing ring 20 may also be comprised of a material adapted to dissolve after an amount of wear of the deodorizing ring 20. While, in the preferred embodiment, the device for deodorizing a sink drain 10 comprises a wear indicator 60, it should be clearly understood that substantial benefit could be derived from an alternative configuration of the device for deodorizing a sink drain 10 in which there is no wear indicator 60.

### STATEMENT OF OPERATION

In order to operate the device for deodorizing a sink drain 10, one places it either directly into the sink drain 12 such that it is directly abutting an interior wall of the sink drain 12, or places it proximate an exterior portion of the sink drain 12. After an amount of use of the device for deodorizing a sink drain 10, the user removes the deodorizing ring 20 or removes the refillable tray 40 in order to replace the deodorizing ring 20.

While the invention has been particularly shown and described with reference to preferred embodiments thereof, it will be understood by those skilled in the art that the foregoing and other changes in form and details may be made therein without departing from the spirit and scope of the invention. For example, although the device for deodorizing a sink drain is shown to be retained by frictional engagement with the interior wall of the drain (see FIGS. 1-12), substantial benefit may be obtained from the device merely resting upon the bottom portion of the sink drain or upon the rubber splash guards found over the garbage disposal. It should also be understood that substantial benefit may be derived from the device being retained above, as well as below, the rubber splash guards. It should be further understood that although the device is shown as being used with a sink drain having a garbage disposal unit, substantial benefit may be obtained from using the device with a sink drain that is not equipped with a garbage disposal unit.

1. A device for deodorizing a sink drain comprising at least one deodorizing ring, said at least one deodorizing ring being dimensioned to be substantially flush with an interior wall of said sink drain while at the same time defining an aperture dimensioned to allow said sink drain to remain substantially entirely open.

2. The device of claim 1 wherein said at least one deodorizing ring having:
an outer diameter of between 8 cm and 9 cm;
an inner diameter of between 7 cm and 8 cm; and
a width of between 0.83 cm and 2.5 cm.

3. The device of claim 2 wherein said outer diameter being approximately 8.5 cm, said inner diameter being approximately 7.5 cm, and said width being approximately 2.5 cm.

4. The device of claim 1 wherein said at least one deodorizing ring being dimensioned such that an outer surface of said at least one deodorizing ring directly abuts said interior wall of said sink drain.

5. The device of claim 1 wherein said at least one deodorizing ring containing at least one of a deodorizing agent and a clog removing agent.

6. The device of claim 5 wherein said deodorizing agent being one of a surfactant and a caustic soda.

7. The device of claim 5 wherein said at least one clog removing agent being one of a bleach and an enzyme.

8. The device of claim 1 wherein said at least one deodorizing ring containing a fragrance.

9. The device of claim 1 wherein said at least one deodorizing ring further comprising a wear indicator adapted to signal a user to replace said at least one deodorizing ring after an amount of wear on said at least one deodorizing ring.

10. The device of claim 9 wherein said wear indicator being a color that fades as said at least one deodorizing ring being diminished by use.

11. The device of claim 9 wherein said wear indicator being a color that gradually changes to another color as said at least one deodorizing ring being diminished by use.

12. The device of claim 1 wherein said at least one deodorizing ring being comprised of a material adapted to dissolve after an amount of wear of said at least one deodorizing ring.

13. The device of claim 1 further comprising at least one refillable tray, said at least one refillable tray being dimensioned to house said at least one deodorizing ring and being dimensioned such that an outer surface of said at least one refillable tray directly abuts said interior wall of said sink drain while at the same time allowing said aperture defined by said deodorizing ring and said sink drain to remain substantially entirely open.

14. The device of claim 13 wherein said at least one refillable tray having:
an outer diameter of between 8 cm and 9 cm;
an inner diameter of between 7 cm and 8 cm; and
a width of between 0.83 cm and 2.5 cm.

15. The device of claim 14 wherein said outer diameter being approximately 8.5 cm and said inner diameter being approximately 7.5 cm

16. The device of claim 14 wherein said width being approximately 2.5 cm and allowing said refillable tray to house up to three deodorizing rings.

17. The device of claim 16 wherein each of said three deodorizing rings having a width of approximately 0.83 cm.

18. The device of claim 13 wherein said at least one refillable tray being constructed of one of a plastic and a metal.

19. The device of claim 2 wherein said at least one deodorizing ring being substantially crescent-shaped and wherein said outer diameter being substantially larger than an inner diameter of said sink drain.

20. The device of claim 19 wherein said outer diameter being approximately 9 cm, said inner diameter being approximately 8 cm, and said width being approximately 2.5 cm.

21. The device of claim 19 wherein said at least one deodorizing ring being substantially flexible so as to allow said at least one deodorizing ring to contract when a first grip end of said deodorizing ring being squeezed together with a second grip end of said at least one deodorizing ring and so as to allow said at least one deodorizing ring to expand when said first grip end and said second grip end are released such that an outer surface of said at least one deodorizing ring directly abuts said interior wall of said sink drain.

22. The device of claim 14 wherein said at least one refillable tray being substantially crescent-shaped and wherein said outer diameter being substantially larger than an inner diameter of said sink drain.

23. The device of claim 22 wherein said outer diameter being approximately 9 cm, said inner diameter being approximately 8 cm, and said width being approximately 2.5 cm.

24. The device of claim 22 wherein said refillable tray being substantially flexible so as to allow said at least one refillable tray to contract when a first grip end of said refillable tray being squeezed together with a second grip end of said at least one refillable tray and so as to allow said at least one refillable tray to expand when said first grip end and said second grip end being released such that said outer surface of said at least one refillable tray directly abuts said interior wall of said sink drain.

25. The device of claim 19 wherein said at least one deodorizing ring comprising:
a first portion, said first portion having a grip end and a hinge end;
a second portion, said second portion having a grip end and a hinge end; and
a hinge, said hinge being coupled to said hinge end of said first portion of said at least one deodorizing ring and being coupled to said hinge end of said second portion of said at least one deodorizing ring.

26. The device of claim 25 wherein said hinge being configured to allow said at least one deodorizing ring to contract when said grip end of said first portion and said grip end of said second portion of said at least one deodorizing ring being squeezed together and configured to allow said at least one deodorizing ring to expand when said grip end of said first portion and said grip end of said second portion of said at least one deodorizing ring being spread apart such that an outer surface of said at least one deodorizing ring directly abuts said interior wall of said sink drain.

27. The device of claim 25 wherein said hinge being one of a living hinge and a locking hinge.

28. The device of claim 22 wherein said at least one refillable tray comprising:
a first portion, said first portion having a grip end and a hinge end;
a second portion, said second portion having a grip end and a hinge end; and
a hinge, said hinge being coupled to said hinge end of said first portion of said at least one refillable tray and being coupled to said hinge end of said second portion of said at least one refillable tray.

29. The device of claim 28 wherein said hinge being configured to allow said at least one refillable tray to contract when said grip end of said first portion and said grip end of said second portion of said at least one refillable tray being squeezed together and configured to allow said at least one refillable tray to expand when said grip end of said first portion and said grip end of said second portion of said at least one refillable tray being spread apart such that said outer surface of said at least one refillable tray directly abuts said interior wall of said sink drain.

30. The device of claim 28 wherein said hinge being one of a living hinge and a locking hinge.

31. A device for deodorizing a sink drain comprising at least one deodorizing ring, said at least one deodorizing ring being dimensioned to be coupled proximate an exterior portion of said sink drain while at the same time defining an aperture dimensioned to allow said sink drain to remain substantially entirely open.

32. The device of claim 31 wherein said at least one deodorizing ring having:
an outer diameter between 9 cm and 10 cm
an inner diameter of between 8 cm and 9 cm; and
a width of between 0.5 cm and 1 cm.

33. The device of claim 31 further comprising a plurality of legs having a first end and a second end, said first end of each said plurality of legs being coupled to a portion of said deodorizing ring and said second end of each said plurality of legs extending substantially downwardly from said at least one deodorizing ring, said second end of each said plurality of legs being dimensioned to couple said at least one deodorizing ring to said sink drain so as to support said at least one deodorizing ring above said sink drain.

34. The device of claim 31 further comprising:
at least one refillable tray, said at least one refillable tray being dimensioned to house said at least one deodorizing ring; and
a plurality of legs having a first end and a second end, said first end of each said plurality of legs being coupled to a portion of said refillable tray and said second end of each said plurality of legs extending substantially downwardly from said at least one refillable tray, said second end of each said plurality of legs being dimensioned to couple said at least one refillable tray to said sink drain so as to support said at least one refillable tray above said sink drain.

35. The device of claim 34 wherein said at least one refillable tray having:
an outer diameter between 9 cm and 10 cm
an inner diameter of between 8 cm and 9 cm; and
a width of between 0.5 cm and 1 cm.

36. A method for deodorizing a sink drain comprising, in combination, the steps of:
providing at least one deodorizing ring, said at least one deodorizing ring having a deodorizing agent and defining an aperture dimensioned to allow said sink drain to remain substantially entirely open; and
positioning said at least one deodorizing ring such that said at least one deodorizing ring being substantially flush with an interior wall of said sink drain.

37. The method of claim 36 wherein said at least one deodorizing ring being dimensioned such that an outer surface of said at least one deodorizing ring directly abuts said interior wall of said sink drain.

38. The method of claim 36 further comprising the steps of:
providing at least one refillable tray, said refillable tray being dimensioned to house said at least one deodorizing ring and being dimensioned to allow said refillable tray and said sink drain to remain substantially entirely open;
inserting said at least one deodorizing ring into said at least one refillable tray; and
positioning said at least one refillable tray containing said at least one deodorizing ring such that an outer surface of said at least one refillable tray directly abuts said interior wall of said sink drain.

39. The method of claim 36 wherein said at least one deodorizing ring contains a clog removing agent.

40. The method of claim 36 wherein said at least one deodorizing ring contains a fragrance.

41. The method of claim 38 further comprising the steps of:
providing a wear indicator coupled to at least one of said at least one deodorizing ring and said at least one refillable tray; and
replacing said at least one deodorizing ring after said wear indicator indicates that said at least one deodorizing ring being in need of replacement.

42. The method of claim 41 wherein said wear indicator being a color wear indicator on said deodorizing ring and adapted to gradually change to another color as said at least one deodorizing ring being diminished by use.

43. The method of claim 41 wherein said wear indicator being a color wear indicator on said refillable tray and adapted to gradually change to another color as said at least one deodorizing ring being diminished by use.

44. The method of claim 36 further comprising the step of providing a dissolvable deodorizing ring adapted to dissolve after an amount of wear on said at least one deodorizing ring.

45. A method for removing clogs from a sink drain comprising, in combination, the steps of:
providing at least one deodorizing ring, said at least one deodorizing ring having a clog removing agent and defining an aperture dimensioned to allow said sink drain to remain substantially entirely open; and
positioning said at least one deodorizing ring such that said at least one deodorizing ring being substantially flush with an interior wall of said sink drain.

46. The method of claim 45 wherein said at least one deodorizing ring being dimensioned such that an outer surface of said at least one deodorizing ring directly abuts said interior wall of said sink drain.

47. The method of claim 45 wherein said at least one deodorizing ring contains a deodorizing agent.

48. The method of claim 45 wherein said deodorizing ring contains a fragrance.

49. The method of claim 45 further comprising the steps of:
providing at least one refillable tray, said refillable tray being dimensioned to house said at least one deodorizing ring and to allow said refillable tray and said sink drain to remain substantially entirely open;
inserting said at least one deodorizing ring into said at least one refillable tray; and
positioning said at least one refillable tray containing said at least one deodorizing ring such that an outer surface of said at least one refillable tray directly abuts said interior wall of said sink drain.

50. The method of claim 49 further comprising the steps of:
providing a wear indicator coupled to at least one of said at least one deodorizing ring and said at least one refillable tray; and
replacing said at least one deodorizing ring after said wear indicator indicates that said at least one deodorizing ring being in need of replacement.

51. The method of claim 50 wherein said wear indicator being a color wear indicator on said deodorizing ring and adapted to gradually change to another color as said at least one deodorizing ring being diminished by use.

52. The method of claim 50 wherein said wear indicator being a color wear indicator on said refillable tray and adapted to gradually change to another color as said at least one deodorizing ring being diminished by use.

53. The method of claim 45 further comprising the step of providing a dissolvable deodorizing ring adapted to dissolve after an amount of wear on said at least one deodorizing ring.

54. A method for deodorizing a sink drain comprising, in combination, the steps of:
providing at least one deodorizing ring, said at least one deodorizing ring containing a deodorizing agent being dimensioned to be coupled proximate an exterior portion of said sink drain while at the same time defining an aperture dimensioned to allow said sink drain to remain substantially entirely open;
providing a refillable tray, said at least one refillable tray being dimensioned to house said at least one deodorizing ring;
providing a plurality of legs having a first end and a second end, said first end of each said plurality of legs being coupled to a portion of said refillable tray and said second end of each said plurality of legs extending substantially downwardly from said at least one refillable tray;
inserting said at least one deodorizing ring into said at least one refillable tray; and
coupling said second end of each said plurality of legs of said refillable tray to said sink drain so that said at least one deodorizing ring being supported above said sink drain.

55. The method of claim 54 wherein said at least one deodorizing ring contains a clog removing agent.

56. The method of claim 54 wherein said at least one deodorizing ring contains a fragrance.

57. The method of claim 54 further comprising the steps of:
providing a wear indicator coupled to at least one of said at least one deodorizing ring and said at least one refillable tray; and
replacing said at least one deodorizing ring after said wear indicator indicates that said at least one deodorizing ring being in need of replacement.

58. The method of claim 57 wherein said wear indicator being a color wear indicator on said deodorizing ring and adapted to gradually change to another color as said at least one deodorizing ring being diminished by use.

59. The method of claim 57 wherein said wear indicator being a color wear indicator on said refillable tray and adapted to gradually change to another color as said at least one deodorizing ring being diminished by use.

60. The method of claim 54 further comprising the step of providing a dissolvable deodorizing ring adapted to dissolve after an amount of wear on said at least one deodorizing ring.
